(19) United States
(12) Patent Application Publication
(10) Pub. No.: US 2022/0367029 A1
(43) Pub. Date: Nov. 17, 2022

(54) Expert Module for Artificial Respiration and ECLS

(71) Applicant: Xenios AG, Heilbronn (DE)

(72) Inventor: Andreas Terpin, Frankfurt am Main (DE)

(21) Appl. No.: 17/771,245

(22) PCT Filed: Oct. 23, 2020
(86) PCT No.: PCT/EP2020/079948

§ 371 Date: Apr. 22, 2022

(30) Foreign Application Priority Data
Oct. 24, 2019 (DE) 10 2019 007 412.2

(51) Int. Cl. G16H 20/40

(52) U.S. Cl. CPC G16H 20/40

(57) ABSTRACT

The present disclosure concerns expert modules for ventilators as well as methods for optimizing operating parameters in medical systems for supporting the physician in deciding settings of the respective device. Accordingly, an expert module is proposed for a ventilator of a patient, wherein the expert module is adapted to continuously receive at least one current vital parameter of the patient, and wherein the expert module includes an evaluation unit. The evaluation unit is adapted to store the vital parameter for a predetermined period of time, to determine a target value of the vital parameter based on a course of the vital parameter and/or predetermined clinical data, and determine a setpoint or setpoint value of at least one operating parameter of the ventilator based on a course of the vital parameter and the target value of the vital parameter and depending on at least two physiological factors of the patient.

### CROSS-REFERENCE TO RELATED APPLICATIONS

The present application is the national stage entry of International Patent Application No. PCT/EP2020/079948, filed on Oct. 23, 2020, and claims priority to Application No. DE 102019007412.2, filed in the Federal Republic of Germany on Oct. 24, 2019, the disclosures of which are expressly incorporated herein in its entirety by reference thereto.

### TECHNICAL FIELD

The present disclosure is directed to expert modules and methods for ventilators and ECLS (extracorporeal life support) systems for patients as well as ventilators, ECLS systems and systems with corresponding ventilators and ECLS systems, e.g., with the purpose to propose optimized operating parameters of the same with regard to vital parameters of the patient.

### BACKGROUND

In serious or advanced lung diseases, such as ARDS, with severe lung damage and insufficient gas exchange, ventilation of the patient may become necessary. This is particularly the case, if the patient's own respiratory capacity is so insufficient that the patient largely fails to breathe. In these cases, breathing must be supported by a ventilator or the patient must even be artificially ventilated, for example using an invasive mechanical ventilator. Such interventions may be performed not only in patients with lung disease, but also in patients with heart disease or during operations or therapies during which the patient is fully anaesthetized or sedated, artificially preventing the patient from breathing spontaneously.

In order to stabilize the patient's condition and improve it in the long term, the operating parameters must be adapted to the patient's vital parameters. The operating parameters are confronted with physiological limit conditions, so that these cannot be set arbitrarily, but only under consideration of potentially dangerous circumstances for patients. Overly increase in respiratory volume can cause mechanical lung trauma, while minor respiratory volume can lead to acidosis, for example. In the clinical context of a treatment, e.g. in an intensive care unit, a large number of critical vital parameters and physiological factors must usually be taken into account, so that maximum care is required when selecting the operating parameter values to be set.

Accordingly, the settings of a ventilator should be regularly adjusted to the patient's condition and vital parameters, and ventilation should be continuously monitored to provide the best possible therapy for the patient and, as a matter of fact, to reduce the risk of adverse effects on the patient's health.

However, the multitude of parameters to be considered and the high complexity of physiological interactions is difficult even for healthcare professionals to grasp, so settings are often made based on known clinical benchmarks that may not provide optimal therapeutic support for the patient. The decision of the physician therefore depends on empirical values.

A therapy with a mechanical ventilator can also be a contraindication for lung transplants, especially since such a therapy increases the morbidity rate, not least because of the danger of a development of infections caused by the ventilator, which therefore tend to affect the patient's condition even more. For patients undergoing life support lung transplantation, shortening mechanical ventilation and accelerating healing is indicated anyway.

In the case of strongly changing vital parameter values of the patient, it is difficult according to the state of the art to adjust the operating parameter values in such a way that physiological limit states or tolerance ranges are not exceeded under any circumstances. Acute changes in vital parameter values and their effects cannot therefore be sufficiently taken into account according to the state of the art, especially in the case of long-term therapy requirements. In a critical condition of the patient, it is not uncommon that in clinical routine none of the ventilator settings can provide a sufficiently safe and effective therapeutic approach.

Therefore, if no improvement is possible, or if a preset operating mode based on clinical benchmarks is not acceptable for the patient's condition, the physician must decide how to proceed.

Extracorporeal membrane oxygenation can be used as an alternative to artificial ventilation of the patient. In such a procedure, also known as ECMO (extracorporeal membrane oxygenation), the respiratory function of the patient is taken over by an external medical device. The pulmonary or lung function or respiration of the patient is thus replaced by such a medical device, whereby the procedure can guarantee oxygenation and in particular carbon dioxide reduction of the blood for days or weeks and thus relieve the lung so that it can heal without exogenous ventilation.

As extracorporeal life support (ELLS) systems, ECMO systems can thus be used, which include a membrane oxygenator and serve as gas exchangers for the patient's blood. Cannulae are inserted into two blood vessels and the blood is continuously pumped through a membrane oxygenator, which replaces the gas exchange in the lungs. Carbon dioxide is thus removed from the blood and blood enriched with oxygen is returned to the patient. The blood can, for example, be taken from a venous access and returned via a venous or arterial access. Oxygenation is then performed, for example, by means of a veno-venous ECMO (VV-ECMO) or a veno-arterial ECMO (VA-ECMO).

Due to the technical and personnel requirements for the use of ECMO, ECMO therapy is currently regularly used in clinical practice only as a ventilation alternative, and often only when ventilation does not promise sufficient improvement of the patient's condition. If critical limits are exceeded, however, it may happen that even a subsequent ECMO therapy cannot sufficiently stabilize the patient's condition. The timely use of ECLS systems, such as ECMO systems, can therefore be crucial for life support.

The parameters of the ECMO therapy continue to be set manually by a trained physician or cardio technician. In the therapeutic decision as to whether to switch to ECMO therapy, the medical personnel must therefore be able to control or take into account the complex operating procedures and other therapy risks. Decision making is also complicated by the complex interactions with or effects of the targeted ECMO therapy on the numerous physiological aspects. It is therefore currently very difficult for the attending physician to maintain a satisfactory balance between the vital parameters of the patient, the operating parameters of the device and the physiological circumstances.

Accordingly, there is a need to further improve ventilation parameters and adapt them to critical patient conditions in order to further improve the therapeutic effect and thus the patient's state of health and to accelerate healing.

### SUMMARY

Certain aspects of the present disclosure relate to systems and methods that enable continuous improvement of operating parameter values.

Accordingly, an expert module is proposed for a ventilator of a patient which is designed to continuously detect at least one current vital parameter of the patient. The expert module includes an evaluation unit adapted to store at least one vital parameter for a predetermined period of time, to determine a target value of the at least one vital parameter based on a course of the vital parameter(s) and/or predetermined clinical data, and to determine a setpoint or setpoint value of at least one operating parameter of the ventilator based on the course of the at least one vital parameter and the target value of the vital parameter and depending on at least two physiological factors of the patient. Furthermore, the evaluation unit is adapted to determine a setpoint or setpoint value of at least one operating parameter of an ECLS system coupled to the patient—based on the course of the vital parameter and the target value of the vital parameter and depending on the physiological factors. Finally, the evaluation unit is adapted to output a signal indicative of the setpoints or setpoint values of the operating parameters.

Automatic determination of the setpoints or setpoint values provides the attending physician with a decision-making aid for making the best possible settings on the ventilator with regard to the patient's condition. At the same time, the expert module according to the disclosure enables the therapeutic integration of ECLS systems, including ECMO systems or extracorporeal membrane oxygenators, whereby the influence of the membrane oxygenator on vital parameters and physiological factors can be taken into account. In this way, a dual or simultaneous therapeutic approach using ventilation and ECMO is made possible without further increasing the individual complexity of each of these therapies through their combination. Manual handling, according to the state of the art, by the medical staff reaches its limits in case of such a combination therapy. At the same time, the automatic determination of the setpoints or setpoint values of the ECLS system enables an improved use of ECMO systems, in accordance with the invention.

The setpoints or setpoint values can be determined at least periodically, but, e.g., also continuously, by continuously receiving or detecting the patient's vital parameter values, so that the operating parameter values can be adapted to the patient's condition at any time. Although in some implementations automatic setting of these values is possible, in some implementations it is left to the physician. In this case, the evaluation unit proposes the setpoints or setpoint values. However, it may be provided that the signal contains an option or a possibility of accepting the setpoints or setpoint values, so that the corresponding operating parameter values can be set automatically after acceptance of the decision proposal by a physician.

The physician is thus provided with a decision aid by suggesting at least one optimized value of an operating parameter that takes into account the most important vital parameters of the patient, the settings of the ventilator and their effects or impact on the most important physiological factors. As described above, selecting the ventilator settings is already a challenge for healthcare professionals. The chosen attitude may be advantageous for one physiological factor of the patient, but contraindicated for the other physiological factor. The only support provided by current state of the art systems is the monitoring of vital parameters and operating parameter values. For example, an alarm function is currently triggered, once tolerance ranges are exceeded, if a current value is detrimental or even threatening for the patient. However, this approach does not allow for optimization, as any such monitoring is based only on a snapshot. The further effects or impact on the development of the patient's condition or the corresponding physiological factors cannot therefore be taken into account according to the state of the art.

In contrast, automatic determination of the setpoints or setpoint values and the consideration of all these factors considerably simplify therapeutic decision-making, relieving the medical staff of the operating and monitoring duties and reducing the occurrence of errors in the settings.

At the same time, the systems and methods described herein can also facilitate the medical decision as to whether to switch to extracorporeal membrane oxygenation or to involve it in emergency therapy, whereby preferred or optimized settings are automatically suggested by the evaluation unit. Since the operation of ECMO systems is already challenging and can only be performed by specifically trained personnel, integration of such systems into a therapy plan, also in combination with a ventilation function, is significantly simplified by the systems and methods described herein and in many places made possible for the first time at all. The controllability of a simultaneous use of ECMO systems together with ventilation thus ensures an improved therapeutic effect and reduces the morbidity rate of patients through faster healing processes.

Alternatively, the automatically determined setpoints or setpoint values can indicate that ventilation of the patient can be continued exclusively with an ECLS system without switching on an extracorporeal life support system or therapy. A decision regarding the setting can therefore be made step by step, whereby the use of the therapeutic systems is first entered or confirmed and the setpoints or setpoint values are then (again) determined and, if necessary, adopted. For example, a physician can manually activate an ECLS system. The evaluation unit automatically recalculates the setpoints or setpoint values of the ventilator and the ECLS system.

The decision support for the physician is provided by outputting a signal, whereby the signal corresponds to the certain setpoints or setpoint values. In other words, the signal may be an indicator of a proposed reduction or increase in a given operational parameter value, for example by means of an optical and/or acoustic indicator. For example, one or more LEDs can be provided on a surface of the expert module, which suggest a corresponding change of a respective operating parameter value by their color or positioning.

The setpoint or setpoint values can be determined by a physiological model that is stored in the evaluation unit and receives the vital parameter values of the patient. The physiological model can determine target values a priori. Thus, the model is not only based on a snapshot, but also includes the course or a development and variation of the vital parameter. Clinical guideline values for the clinical picture of the respective patient are preferred. Furthermore, the effects or impact of an operating parameter value on at least two physiological factors of the patient are considered. For example, a correlation can be established with previously collected patient data stored in a database, i.e., from a patient collective with comparable clinical pictures and vital parameter values and/or by comparison with test series and experimental values. A learning algorithm can also be used which, for example, also takes into account the patient-specific course of the disease.

For the treatment of the patient, different gas mixtures can be provided as respiration gas, so that beside air of the natural composition for example medical gas mixtures with an enrichment of oxygen, nitrogen oxide, helium, carbon monoxide, and/or one or more fogged medicines can be provided. The physiological model may be such that the effects or impact of the composition of the medical gas mixture on the physiological factors and vital parameters can be taken into account or, conversely, a proposal for an appropriate selection of the gas mixture supplied can be made.

The expert module, which serves as decision support and can be configured as a monitoring module for at least one vital parameter and/or the operating parameter values, is a modular unit that can be implemented in a ventilator or an ECMO console, for example, or can be communicatively coupled to it as a separate unit. It may also be provided that the expert module can be communicatively coupled with a monitor or an external device, such as a portable (e.g., wireless) device of the medical staff and/or a central monitoring system. Vital parameters, and, e.g., the operating parameter values for several ventilators and/or ECLS systems, can be easily and even externally monitored, for example by means of an integrated communication module. It may also be provided that such a communication module feeds depersonalized data, such as the received vital parameters and the calculated setpoints or setpoint values, into a central physiological learning module in order to enable an enlargement of the data set and an improvement of the physiological model. Such data can, for example, be sent via a server or cloud and/or be stored there and, if necessary, be processed.

The expert module is also preferably adapted to receive actual values of the operating parameters from the ventilator and the ECLS system, whereby the evaluation unit is adapted to determine the setpoints or setpoint values as a function of the actual values. In this way, it can be evaluated whether the preferred settings match the current settings or whether the operating settings or operating parameter values should be adjusted to optimize the therapy. Thereby, feedback is provided when and to the extent that the proposed operating parameter values have been adopted; thereby, it is also allowed for checking whether the calculated or modeled effects or impact on physiological factors and/or vital parameters can be adequately represented.

The actual values can be received continuously via an interface. One or more vital parameters can also be received via such an interface, for example via a communicative connection with an external measuring device or with integrated measuring devices of the ventilator or the ECLS system, if the expert module is implemented in a ventilator or an ECLS system.

The ventilator may be configured to support spontaneous breathing of the patient or to provide artificial respiration of the patient. After invasive introduction into the respiratory tract, mechanical ventilators can support respiration or replace the patient's respiration, if necessary, e.g., via endotracheal intubation or tracheostomy. In intensive care units, such measures are performed in about 10 to 15% of ARDS patients. However, it is known that about 20% of these patients would actually need such a therapy.

The operating mode of the ventilator may depend on the patient's condition, but the ventilator may be supportive, for example, to allow weaning or follow-up care. If the ventilator is switched to a controlling operating mode, completely artificial ventilation is carried out. In this operating mode, breathability is provided by the ventilator alone, if spontaneous breathing of the patient is absent or minimal As a rule, the patient is sedated so that spontaneous breathing is no longer necessary. Too high or prolonged sedation, however, carries risks, so the target dose is usually set to keep the patient as awake as possible, but spontaneous breathing by the patient is not excluded.

In severe indications, such as ARDS, this possibility for sedation often does not exist. Nevertheless, attempts should be made to reduce the duration of artificial ventilation as far as possible. A suitable modus operandi is made possible by the combination of ventilation and ECLS or ECMO therapy according to the invention. By suggesting the best possible therapy for the patient, the condition and healing of the patient can be improved and the basic goal of shortening the duration of therapy can be achieved. The expert module or evaluation unit can assist in decision making whether a combination therapy with the aid of a ventilator and, for example, an ECMO system is indicated or whether monotherapy can provide a more promising therapeutic outcome with the ECMO system or ventilator, respectively, only. For therapy, assistance is provided in selecting the operating parameters of the ECMO system and/or ventilator. In the case of the ECMO system, the operating parameters concern in particular the setting of the blood flow (in the form of the speed setting or 1/m specification) and the setting of the gas flow (1/min) for the gas exchanger. Operating parameters of the ventilator to be set can be, for example, inspiration pressure, inspiration time and expiration time.

In some embodiments, the expert module is coupled with a monitor, whereby the output of the signal includes a graphic reproduction or representation of the setpoint or setpoint value, the actual value, the target value, and/or the course of the corresponding value. For example, the monitor can be a monitor or a user interface of a ventilation system or ventilator, wherein the expert module is coupled to or implemented in the ventilator. Alternatively, the expert module can also be implemented as a separate modular unit or in an ECMO console with a monitor. By displaying the preferred setpoints or setpoint values, medical personnel can immediately track which operating parameter values of the ventilator could or should be changed to stabilize or improve the patient's condition.

By displaying the actual value, a comparison with the current settings is also possible, so that the staff or the physician can immediately recognize which deviation of the proposed setpoint or setpoint value from the current actual value occurs. If, for example, this deviates only slightly, the physician can decide whether a minor adjustment would be required or vice versa, whether no further adjustment would be required. On the other hand, a larger deviation can cause the physician to re-examine the patient's condition and, in particular, the settings and functionality of the ventilator and the ECLS system.

Finally, the display of the target value offers the possibility of illustrating the effects or the impact on the vital parameter in an understandable way, whereby the target value can optionally be changed or entered manually. The presentation of the course of the disease offers a further decision-making aid, whereby the physician obtains an overview of the course of the disease or the condition of the patient and can intervene accordingly—in the event of deterioration or no improvement—by adjusting the proposed setpoints or setpoint values.

The evaluation unit can determine the setpoints or setpoint values, e.g., from a physiological model stored in the evaluation unit, and the signal may include a graphical representation of an effect of the setpoints or setpoint values on the physiological factors modeled by the evaluation unit. The physiological model described above can predict the effects or the impact on physiological factors using an assessment function, for example by choosing a scale between a negative range and a positive range or using any other assessment scheme. For example, a negative effect can be represented by a negative value, a neutral or stability-maintaining effect by a value around zero, and a prognosis improving the patient's condition by a positive value. Such an evaluation can alternatively or additionally—also by means of a (graphical) representation—represent, e.g., opposite (i.e., a predicted improvement of a physiological factor with simultaneous predicted deterioration of another physiological factor) physiological factors. For example, if more than two physiological factors are taken into account, a polygon can be mapped whose marginal area corresponds to a negative effect and whose central area corresponds to a positively predicted effect. The different impact predictions can be represented as points in the polygon. To facilitate and simplify the interpretation by the physician, the points can optionally be connected as a polygon.

The respective physiological factors can, as mentioned above, also develop differently from each other, so that an adjustment of the operating parameter value, for example, slightly improves one factor, while another factor can thereby move into a critical range. These developments of individual factors can also be taken into account by the graphic representation, so that the mental coping with complex connections as a result of numerous variables no longer has to be performed by the physician and the decision can be facilitated thereby. In contrast to a representation of the relevant parameters on different devices and the output of alarm signals in the event of deviations from individual values, all decision-relevant data can be directly viewable on a central monitor and visible in the context of the course of the patient's condition.

It may be possible to show the effects of the settings separately for the ventilator and the ECLS system. In addition to the predicted effects of the setpoints or setpoint values, a graphical representation of the modeled effect of the actual values on the physiological factors, for example, can be performed. By such a graphical representation, for example by means of polygons, the physician or the medical personnel can recognize immediately for which factors an improvement is to be expected, e.g., by comparison of the actual condition with the calculated and/or suggested condition. Furthermore, the representation of an actual condition without the use of an ECLS system and the representation of the proposed condition after the use of an ECLS system enables the physician to immediately recognize the expected effect, e.g., of a gas exchanger function, on the condition of the patient. Displaying the setpoints or setpoint values for the ECLS system can simplify its implementation, making it easier for the physician to make a decision.

Although the physiological factors include a variety of patient-relevant factors, which typically means that they are not operating parameters of the ventilator, these are primarily related to the patient's pulmonary or lung function, respiratory system and/or cardiovascular system. Accordingly, the physiological factors are preferentially indicative of an over-ventilation or a ventilatory insufficiency. For example, the physiological factors may be selected from the group including mechanical pulmonary trauma, atrophy, barotrauma, volutrauma, alkalosis, oxygen toxicity, absorption alectase, acidosis, hypoxia, stress, and hemodynamic side effects. In one embodiment, none of the at least two physiological factors is the patient's blood pressure. One or more of these physiological factors may be represented.

For example, one or more operating parameters can be optimized to reduce breath volume and peak inspiratory airway pressure to prevent lung trauma. On the other hand, acidosis should be avoided, so that the ventilation pressure should be adjusted only slightly, without increasing the risk of developing lung trauma. The calculated effects on the physiological factors can optionally be continuously improved in their prognostic reliability by feedback and corresponding calculation with the current vital parameters received from the patient. It may be optional for the expert module or evaluation unit to request the measurement of at least one additional vital parameter to improve the result of feedback optimization.

As the patient's condition is usually characterized by many vital parameters, which are associated with a correspondingly large number of physiological factors, the evaluation unit can be adapted to determine the setpoint or setpoint value depending on three or more, e.g., five or six physiological factors. Preference shall be given to at least one of the factors from the group of mechanical pulmonary trauma, atrophy, oxygen toxicity, acidosis, hypoxia or low oxygen saturation, and/or stress.

As described above, physiological factors can influence each other, so that an improvement of one factor may cause a deterioration of another factor. The evaluation unit can be adapted to determine the setpoint or setpoint value using a physiological model stored in the evaluation unit in such a way that negative effects on all the physiological factors considered are minimized and/or the physiological factors are not exceeded in any case after changes to the respective specified or predetermined tolerance ranges.

It will be observed on a regular basis that although no optimal conditions for all of the respective factors are achieved, the setpoints or setpoint values will improve the vital parameters and physiological factors without exceeding the tolerance ranges of the individual factors. Thus, although the effects on one factor may be less beneficial therapeutically, such a circumstance may be individually and case-by-case acceptable in terms of the resulting overall improvement (by improving other factors). One or more factors can also be improved in a targeted manner, but the evaluation unit can determine the setpoint or setpoint value in such a way that this does not lead to the tolerance range of the other factors being exceeded.

Physiological factors can also be weighted differently, depending on the patient's condition or preferred treatment, so that one or more factors can be given special consideration when determining the setpoint or setpoint values. It will still be possible to supplement the therapy by the (proposed) activation of an ECLS system, especially since ventilation parameters and conditions can thereby be avoided which, in the critical condition of the patient—without the use of an ECLS system—could in turn endanger the patient. If necessary, ventilation can be reduced or switched off by switching on an ECLS system, so that it is also possible to switch to an ECLS system.

The weighting may also be variable so that the physician can adapt the physiological model to the treatment method and the patient. In some embodiments, it may be provided that the physician can manually change the points in the graphical representation, whereby the physiological model or evaluation unit determines the required setpoints or setpoint values and, if necessary, indicates whether the setpoints or setpoint values exceed physiological limit states. This allows the physician to compare different alternatives and the effects of any deviations from the proposed setpoints or setpoint values, thus providing further support in the physician's decision making process.

The vital parameters can be provided directly by measuring devices communicatively coupled with the expert module and received by the evaluation unit, for example if the expert module is implemented in a ventilator, or the expert module can also be coupled with other external devices. The at least one vital parameter can be selected from a list including pulsoximetric oxygen saturation, expiratory oxygen fraction, expiratory carbon dioxide fraction, oxygen uptake capacity, carbon dioxide release and blood pH.

In order to increase the accuracy of the setpoint or setpoint value to be determined, the expert module can be adapted to receive at least two vital parameters, e.g., at least three or four vital parameters.

Further, the at least one operating parameter of the ventilator can include the respiratory volume, the peak inspiratory pressure, the positive end expiratory pressure, the respiratory frequency, the inspiratory oxygen fraction, the inspiratory carbon dioxide fraction, and/or the ratio between the inhalation duration and the exhalation duration. And the at least one operating parameter of the ECLS system can include the blood pump flow rate, the gas volume flow, and/or the system pressure.

The positive end expiratory pressure, also known as PEEP (“positive end-expiratory pressure”) serves to counteract a reduction in the residual functional capacity and a collapse of the alveoli, i.e., the formation of atelectasis. An increase can have a negative effect through overstretching of the ventilated areas of the lungs, reduced cardiac output, and/or increased intercranial pressure. Nevertheless, an increase in the positive end-expiratory pressure, especially in ARDS patients, may reduce their mortality rate. In the state of the art, protocols are provided for setting the pressure. However, these recorded tables do not take into account the different individual respiratory mechanics and thus offer only an unreliable decision-making aid for the treating physician. However, the evaluation unit described herein takes into account not only the predicted effects on the various physiological factors, but also the influences of the use of a connected ECLS system, so that the proposed setpoint or setpoint value can take into account the overall system with its numerous variables.

Furthermore, by adjusting the inspiratory oxygen fraction (FiO₂), a hypoxic state of the patient can be prevented, whereby the fraction should generally be adjusted restrictively in order to avoid oxygen toxicity, for example. For example, the oxygen fraction can be adjusted so that an arterial oxygen saturation of between about 80-95 percent or an arterial partial pressure of the oxygen of between about 50 and 90 mmHg is achieved.

Too high a respiratory volume can cause lung trauma due to overstretching, so that the respiratory volume can be set at no more than 6 mL/kg of the patient's weight, for example in ARDS patients. However, for patients not suffering from ARDS, this may be increased to up to 8 mL/kg of patient weight.

Furthermore, in order to avoid barotrauma, for example, the inspiratory peak pressure can be adjusted so that the peak pressure is preferably below 30 cm H₂O.

To best assist healthcare professionals with the variety of ventilator and/or ECLS system settings, the evaluation unit can be configured to determine a setpoint or setpoint value for two, three, or four ventilator operating parameters and a setpoint or setpoint value for two ECLS system operating parameters. Setpoint or setpoint values for the inspiratory oxygen fraction, respiratory volume, positive end expiratory pressure and respiratory frequency can suggest optimized settings to the physician that directly affect physiological factors.

The evaluation unit can be adapted to determine the setpoint or setpoint value in dependence on a ratio of (i) ventilator support to (ii) ECLS support in terms of vital signs, wherein the ratio is determined by the evaluation unit. For example, the setpoint or setpoint value can cause the ECLS system to provide about 70 percent of the maximum oxygen uptake, if respiration is to be supported about 70 percent by the ECLS system and about 30 percent by the ventilator.

An adjustment of the setpoint or setpoint values and/or a change in the support ratios of the ventilator and ECLS system can also make it reasonable to measure certain vital parameters in order to enable sufficient monitoring and continuous optimization of the setpoints or setpoint values. Accordingly, it may be provided that the signal continues to include a requirement to receive another, preferably specific, vital parameter.

The evaluation unit may also be adapted to compare the course of the vital parameter with a tolerance range and/or a modeled course and to output a signal, including an alarm function, if a deviation of the vital parameter exceeding a predetermined threshold or limit value is detected. Instead of outputting an alarm signal, once a current actual value is exceeded, an alarm signal is only output in the event of an overall negative tendency in the patient's condition.

An adjustment of the operating parameter values is therefore only necessary if there is an overall destabilization of the patient condition. For example, a peak value of a vital sign may exceed a tolerance range but return to normal for a short time, so that no adjustment of the corresponding operating parameter value is required. Thus, according to the invention, an intelligent monitoring of the patient is possible, which largely limits the time-consuming control by the physician and at the same time even suggests a corresponding, certain setpoint or setpoint value for the operating parameter, in order to improve the condition of the patient.

The above object is further solved by a ventilator, which includes the inventive expert module. The ventilator is preferably a mechanical ventilator. Accordingly, the ventilator can provide invasive or non-invasive ventilation to the patient, either supporting or artificially replacing the breathing, with the degree of support variable during treatment.

In this case, the expert module can be integrated into the ventilator, so that a monitor of the expert module can, for example, be designed as a user interface of the ventilator. Furthermore, an interface can be provided for receiving vital parameters and/or the ventilator can include a device for recording at least one vital parameter of a patient which is communicatively coupled with the expert module.

The above object will continue to be solved by an ECLS system, which includes the inventive expert module. The ECLS system can be an ECMO system. Accordingly, the ECLS system can provide extracorporeal membrane oxygenation of the patient's blood, with pulmonary or lung function or respiration supported or artificially replaced by the ECLS system. The ECLS system can include a console with a monitor, wherein the monitor is adapted to map the signal.

Furthermore, a system is proposed that includes a ventilator and an ECLS system. The components of the expert module, the ventilator and the ECLS system can thus be adapted to each other so that redundancy is largely eliminated and the system can be designed more compactly. This also allows direct control of both the ventilator and the ECLS system to be implemented. A central communication interface can be provided, whereby the relevant components of the system can be adjusted and adapted by the expert module.

In certain aspects, a method for monitoring vital parameters of a patient includes at least the following steps:
Continuous reception of at least one current vital parameter of the patient from an expert module;
Storing at least one vital sign for a predetermined period in the expert module;
determining a target value of the at least one vital parameter based on a course of the at least one vital parameter and/or predetermined clinical data in the expert module;
determining a setpoint or setpoint value of at least one operating parameter of a ventilator to be provided for the patient in the expert module, wherein the setpoint or setpoint value is determined based on a course of the at least one vital parameter and the target value of the at least one vital parameter and depending on at least two physiological factors of the patient;
determining a setpoint or setpoint value of at least one operating parameter of an ECLS system to be provided for the patient in the expert module, wherein the setpoint or setpoint value is determined based on the course of the at least one vital parameter and the target value of the at least one vital parameter and depending on the physiological factors; and
outputting by the expert module of a signal characterizing the set values of the operating parameters.

The method can be implemented and executed in an expert module, whereby the individual parts are executed by an evaluation unit integrated in the expert module. The method serves as a decision-making aid for a physician to enable an advantageous and best possible therapy for the patient.

The method can be advantageous for patients with respiratory insufficiency, wherein the method supports the physician in weighing up the medical device or system to be used.

In some embodiments, the signal may indicate a proposed use of the ventilator and/or ECLS system, such as an ECMO system. Accordingly, it can be optionally specified first whether a medical device should be used or switched on or whether it should be switched to another medical device. The signal can then or simultaneously index specific operating parameter values of the device or system to assist the clinician in making and/or adjusting settings and optimize the operating parameter values to improve physiological factors and/or vital signs. The signal and the setpoints or setpoint values are therefore modelled values, whereby a large number of factors and variables are taken into account. These are suggested to the physician to facilitate a therapeutic decision and commissioning or adjustment of settings.

The integration of further or alternative medical devices or systems is thereby considerably facilitated, whereby the decision support can be carried out accordingly step by step. Furthermore, the setpoints or setpoint values are also determined on the basis of the at least one received vital parameter, so that fluctuations or changing values are taken into account when determining the setpoints or setpoint values. In some embodiments, the target value and/or the setpoints or setpoint values are determined continuously or periodically.

It is also possible for the expert module to determine the target value and/or the setpoints or setpoint values again after manual setting of the operating parameter values. Thus, a feedback or feedback loop is provided, whereby the method can optionally provide that the effects of the settings on the physiological factors and/or the at least one vital parameter are fed into a physiological model or into a learning algorithm.

To indicate to the physician whether further optimization of the operating parameter values or of the medical devices or systems used in therapy is possible, the expert module can continue to receive actual values of the operating parameters from the ventilator and/or the ECLS system, whereby the setpoints or setpoint values are determined as a function of the actual values. Thus, a comparison between the actual state and a calculated setpoint state is carried out, whereby the signal indicates whether a further therapeutic improvement would be possible based on the theoretically calculated effect on the physiological factors.

Although the signal, as described above, can be output in the form of one or more LEDs, for example, as an indicator of a suggested reduction or increase in a certain operating parameter value, a more detailed or more concrete visual representation of the setpoints or setpoint values in many cases facilitates the physician's decision. This enables a comparison with, for example, standard or guideline-compliant operating parameter values. Accordingly, the signal can be output as a graphical representation of the setpoint or setpoint value, the actual value, the target value, and/or the course of the corresponding value(s) on a monitor communicatively coupled with the expert module.

The setpoints or setpoint values can also be determined using a physiological model stored in the expert module, wherein the signal can include a graphical representation of an effect or impact of the setpoints or setpoint values on the physiological factors modeled by the expert module. In some embodiments, the reproduction of the effect is separatel for the ventilator and the ECLS system and/or it includes the signal, in addition to the effect of the setpoints or setpoint values, a graphical reproduction representation of a modeled effect of the actual values on the physiological factors.

As described above, the physiological model can predict the effects on physiological factors by means of an evaluation function, whereby, for example, a negative effect can be characterized by the representation of a negative value, a neutral or stability maintaining effect with a value around zero, and a prognosis improving the patient's condition with a positive value.

Accordingly, it may be provided that the setpoints or setpoint values are determined using a physiological model stored in the expert module in such a way that a negative effect on the physiological factors is minimized and/or the physiological factors do not exceed a predetermined tolerance range.

For example, physiological factors may be characteristic of over-ventilation or ventilation insufficiency. Thus, when determining the setpoints or setpoint values, physiological factors can be taken into account which are opposite, i.e., a predicted improvement of one physiological factor can simultaneously cause a predicted deterioration of another physiological factor. Such opposite effects can optionally be represented in a graphical representation so that, for example, if more than two physiological factors are taken into account, a polygon can be represented whose peripheral area corresponds to a negative effect and whose central area corresponds to a positively predicted effect, with each corner corresponding to a physiological factor and the respective impact predictions being represented as points in the polygon. To facilitate and simplify the interpretation by the physician, the points can optionally be connected as a polygon. Thus, the effect of the proposed setpoint or setpoint values on the respective physiological factors is simplified and visible at a glance.

If the signal indicates that a combination of therapy with a ventilator and an ECLS system, for example an ECMO system, is recommended, the method may further provide that the setpoints or setpoint values are determined as a function of a ratio, determined by the expert module, of the ventilator's support intensity to the ECLS system's support intensity relative to the at least one vital parameter.

The signal may also include a requirement to receive at least one other, e.g., specific, vital parameter. For example, to enable an ECMO system, it may be necessary to measure and provide specific blood values and/or it may be advantageous for the calculated proposed setpoints or setpoint values to consider additional vital signs in order to further optimize the setpoints or setpoint values for the physiological factors using the at least one additional vital sign.

Furthermore, the course of the vital parameter can be compared with a tolerance range and/or a modeled course in the expert module, whereby an alarm signal is issued if a deviation of the vital parameter is detected which exceeds a predefined threshold value or limit value. Alternatively, or in addition, the expert module may issue an alarm signal, once a manual setting of operating parameter values exceeds a specified or predetermined threshold or limit of operating parameter values, at least one vital parameter, and/or the modeled effect on physiological factors. Thus, the physician can be made aware of a systemic error and/or a physiologically critical condition as quickly as possible or a potential physiological risk can be pointed out if a manual adjustment could endanger the patient.

Although the method is not limited to the expert module described herein, the method can be carried out with the expert module described herein. Likewise, the various aspects of the expert module described above may be implemented in the method without the method being limited to a structural design of the expert module, unless they have already been explicitly described with regard to the method.

### BRIEF DESCRIPTION OF THE FIGURES

Specific embodiments are explained in more detail in the following description of the figures.

FIG. 1 is a schematic representation of an implementation of an expert module on a logical level;

FIG. 2 is a schematic representation of a physiological model stored in an evaluation unit;

FIGS. 3A to 3C show alternative designs of systems with an expert module together with a communicatively coupled ventilator and ECLS system; and

FIGS. 4A and 4B show a history/course, vital signs values and suggested specific setpoints or setpoint values of operating parameters of the ventilator and ECLS system.

### DETAILED DESCRIPTION

In the following, embodiments are described on the basis of the figures. The same, similar or equivalent elements in the different figures are provided with identical reference signs, and a repeated description of these elements is omitted in order to avoid redundancies.

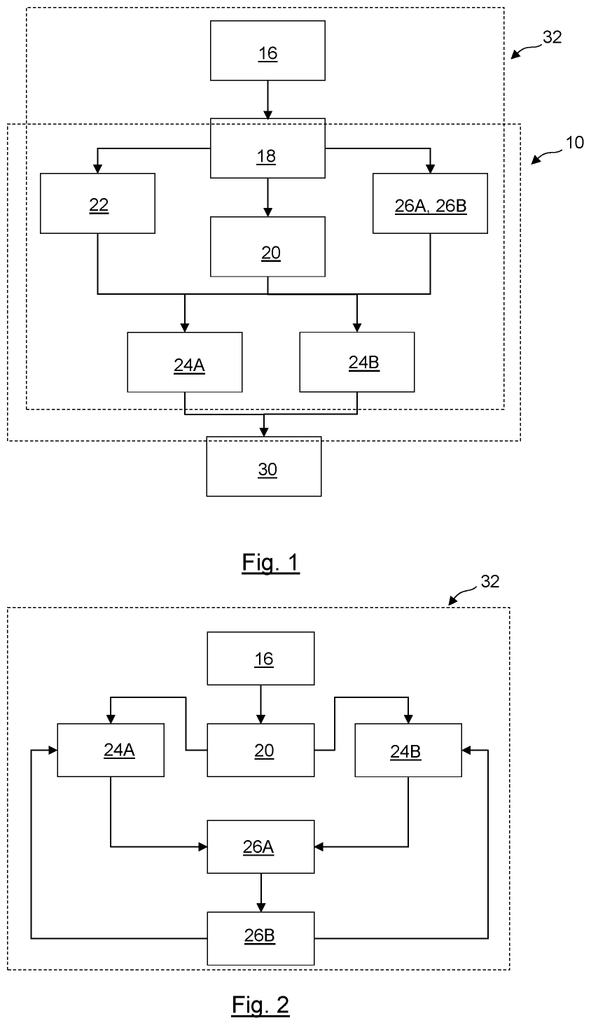

FIG. 1 schematically depicts an expert module 10 for a patient's ventilator, which is essentially an implementation at a logical level, as indicated by the dashed line. It is therefore a logic that can be executed, for example, by a microprocessor provided in the expert module 10 and which is stored in a corresponding memory. As input signal for the logic, at least one vital parameter 16 is received via a non-displayed interface from an evaluation unit 18 present in the expert module 10. The evaluation unit 18 forms a central component and can be integrated as a hardware and/or program module in the expert module 10. For example, evaluation unit 18 may be implemented as part of a control unit of expert module 10, if expert module 10 is optionally designed as part of a ventilator and/or as a controller for the ventilator. However, it may also be provided that expert module 10 is designed as part of an ECLS system or as a separate unit communicatively coupled to a ventilator and/or an ECLS system, such as an ECMO system.

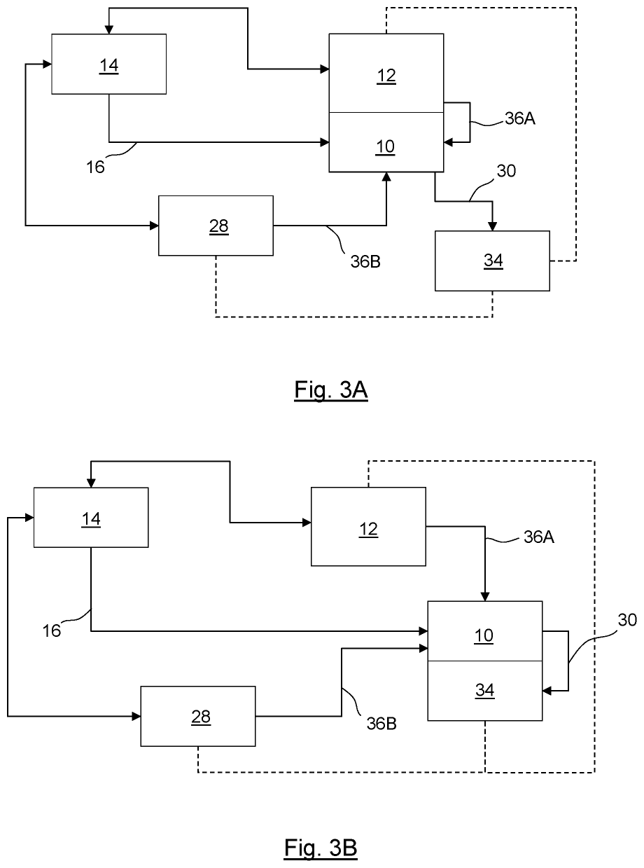

The evaluation unit 18 processes the at least one vital parameter 16 and determines a target value 20, e.g., based on clinical benchmarks, as shown by the corresponding arrows. Furthermore, the vital parameter 16 is continuously received and stored, so that a course 22 of the vital parameter 16 can be taken into account when determining the target value 20. Based on course 22 and target value 20, a setpoint or setpoint value of 24A is predicted for a ventilator operating parameter that should improve the patient's physiological condition. In determining this value, the effects or impact of the setpoint or setpoint value on at least two physiological factors 26A, 26B are also taken into account, so that when optimizing the respective operating parameter and at least one physiological factor 26A, 26B, possible negative effects or impact on another physiological factor 26A, 26B are minimized, as will be explained below with respect to FIG. 2.

For example, the current maximum oxygen intake on the patient can be measured as vital parameter 16. This parameter can be improved by increasing the respiratory volume. However, the increase in respiratory volume may also result in the risk of mechanical lung trauma as a result of overstretching. In order to avoid or reduce this predicted negative effect, the operating parameter value for the respiratory volume, for example, can only be increased up to a tolerance limit for the other physiological factors. Thereby, any risk to the patient is largely excluded a priori.

Furthermore, expert module 10 is coupled to an (not shown) ECMO system including an extracorporeal membrane oxygenator and may include a console, whereby such a system is required not only for specific clinical pictures of the patient and critical patient conditions, but generally for improvement of physiological factors 26A, 26B. The handling of an ECLS system or ECMO system as such is already challenging for the operating personnel. However, if it is to be connected to parallel ventilation, further operating parameters for the ECMO system must be taken into account in addition to the vital parameters 16 and the operating parameters of the ventilator. This is likely to be a significant entry barrier even for qualified medical professionals to the use of a combination therapy of membrane oxygenation or ECMO and ventilation. The operating personnel must therefore first decide whether a parallel combined therapy of ECMO and ventilator should be carried out at all or whether ventilation should be carried out alone or ECMO alone. For example, conditions can provide for the use of an ECMO system with an extracorporeal membrane oxygenator as a replacement for ventilation of the patient, i.e., the ventilator can then be switched off. However, the operating parameters must be determined taking into account the physiological factors for each therapy.

However, expert module 10 also takes into account the connected ECLS system, for example an ECMO system, wherein all control parameters for the ECMO system or ECMO therapy converge in an ECMO console. This also allows the determination of at least one setpoint 24B of the ECLS system, such as blood flow for the blood pump and/or gas flow for the gas blender, in addition to the ventilator. This is made possible by a physiological model 32, which is marked with a dashed line.

Evaluation unit 18 therefore determines not only at least one 24A setpoint for the ventilator, but also at least one 24B setpoint for the ECLS system. These setpoints or setpoint values 24A, 24B can be transmitted to the physician or the medical staff by means of a corresponding signal 30, for example as a graphic representation of the setpoint or setpoint value 24A, 24B for the respective operating parameter. This provides the physician with appropriate decision support so that a therapeutically sensible setting of the ventilator and the ECLS system is made easier or even possible in the first place, even with the large number of variables and factors to be taken into account.

FIG. 2 shows the physiological model 32 schematically. As described above, the course is recorded from the received vital parameter 16 and a corresponding target value 20 is determined. A preferred setting of the ventilator and the ECLS system is then calculated from the target value 20 and the history or course (not shown), so that corresponding setpoints or setpoint values 24A and 24B are determined. For each setpoint or setpoint value 24A, 24B, the effects on at least one physiological factor 26A are calculated or modelled, whereby the effect can be based on an evaluation on a scale. For example, the scale may include a minimum negative value and a maximum positive value, wherein a neutral effect, i.e., neither a deterioration nor an improvement in the physiological factor, is zero.

The physiological model 32 also provides that after determining a (maximum) positive effect on the one physiological factor 26A, the effects on at least one other physiological factor 26B are calculated or simulated or modelled, as shown by the corresponding arrows. If the predicted effects are negative and exceed, for example, a tolerance range, the setpoint or setpoint value of 24A, 24B is determined again. The adjustment of the setpoint or setpoint value 24A, 24B takes into account the effect on the further physiological factor 26B in such a way that it is within a tolerance range. Since the physiological factors 26 can be mutually dependent, it may be provided that only one of the physiological factors 26A, 26B can be improved by the corresponding setpoint or setpoint value 24A, 24B, whereby only a slight improvement, stabilization or even a more or less minor deterioration of the other physiological factor 26A, 26B is accepted as inevitable.

This process can be iterative. The nominal values 24A, 24B are entered continuously and with dynamic values. The effects or impact on physiological factors 26A, 26B are used as feedback. For example, a first setpoint or setpoint value of 24A may have a positive effect on a physiological factor of 26A (e.g., a rating of 5 on a scale of −10 to 10, for example), while this choice may also have a negative effect on another physiological factor of 26B with a rating of −4, for example. It is possible that this negative value should not exceed the tolerance range. In a second iterative calculation, however, the setpoint or setpoint value 24A is reduced by, for example, 20 percent. Thereby, a reduction of the first physiological factor 26A to only 4 is caused, but an improvement of the further physiological factor to 0 is achieved. The effects or impact on the physiological factors 26A, 26B can—as a matter of fact—be linear or non-linear. Thus, this setpoint or setpoint value of 24A can result in an overall improvement of the physiological condition of the patient.

However, a third iterative calculation with a further adjustment or reduction of the setpoint or setpoint value 24A could improve both the first and the further physiological factor 26A, 26B, which could shorten the patient's recovery process in the long term. However, in some embodiments, it may not make an automatic setting, but only suggest a setpoint value of 24A, 24B, so that the physician has to make the setting on his own, but is supported in the decision making for the setting. Thus, the setpoint or setpoint value 24A, 24B can be adopted or deviated from, e.g., to concretely improve a specific physiological factor 26A, 26B, e.g. if an acute deterioration of such a factor should occur. The physician thus only needs to oversee a few operating parameters and factors and is further supported by the suggested 24A, 24B setpoints when making or adjusting settings on both the ventilator and the ECLS system. However, although not primarily notified, full automation of the setting procedure is possible.

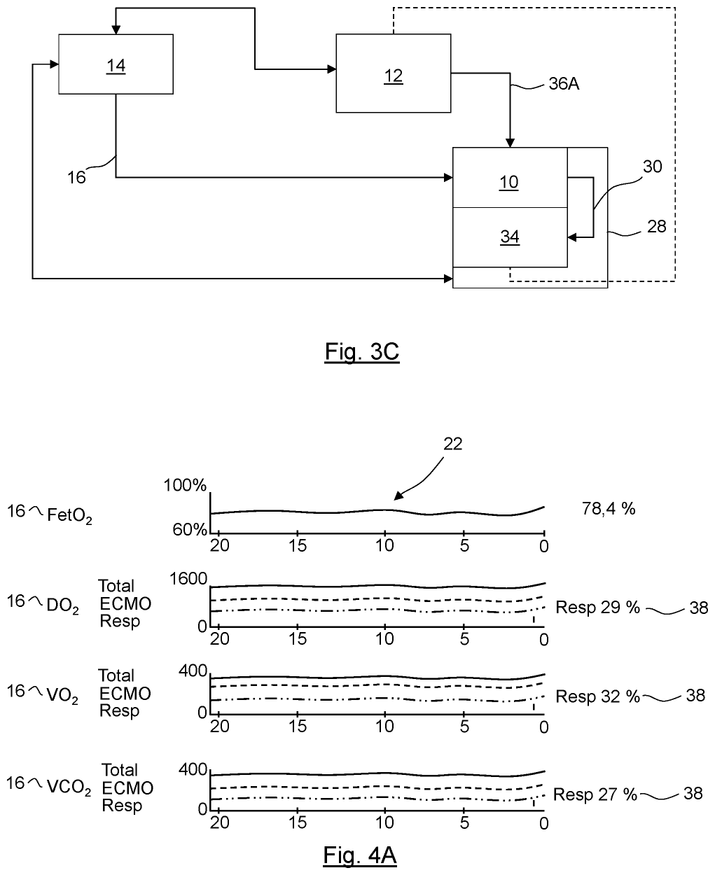

FIG. 3A shows a system with an expert module 10 implemented in a ventilator 12 and a coupled extracorporeal membrane oxygenator 28, whereby a patient 14 is treated simultaneously by ventilator 12 and an ECLS system 28. However, as described above, this configuration is optional only and it may also be provided that the expert module 10 is designed in the ECLS 28 system or as a separate unit coupleable to the ventilator 12 and the ECLS 28 system. For example, the ventilator 12 can be a mechanical ventilator that is invasively connected to the patient's airways via endotracheal intubation. The ECLS system 28 is an ECMO system that can, for example, be connected to the patient's circulation by means of two cannulae, whereby the blood is taken, for example, from a venous access and returned via a venous or arterial access. Extracorporeally, the blood is continuously pumped through a membrane oxygenator, which replaces the gas exchange in the lungs, so that carbon dioxide is removed from the blood and oxygen-enriched blood is returned to the patient. As shown by the corresponding arrows, patient 14 is treated simultaneously with both ventilator 12 and ECMO System 28.

The expert module 10 in this version is integrated in the ventilator 12. However, it may also be provided that the expert module 10 is designed as a separate or external device or integrated into another device. The expert module 10 receives, as described above, at least one vital parameter 16 of the patient 14, so that the evaluation unit (not shown) can prognostically determine and propose a setpoint or setpoint value for both the ventilator 12 and the ECLS system 28, for example by means of a physiological model, in order to improve the physiological condition of the patient 14. These setpoints or setpoint values are output by means of a corresponding signal 30, present on a monitor 34, which enables a graphic representation of the setpoints or setpoint values present in the signal 30 as well as the actual values 36A, 36B received by the ECMO system 28 and the ventilator 12.

Monitor 34 is also optionally displayed as an external unit, but can also be integrated into the ventilator 12 or into a console of the ECLS system or ECMO system 28, for example as part of a user interface. Monitor 34, which is optionally designed as the user interface of a control module, is also configured to make and adjust settings for ventilator 12 and ECMO system 28, as shown by the dashed lines. The monitor 34 thus enables the physician to be assisted in setting the life support devices by displaying the setpoints or setpoint values and adjusting the operating parameter values at one point, eliminating the need for complex calculations by the physician and further preventing the physician from moving back and forth. This approach reduces the physician's cognitive effort in setting up the device and allows the best possible operating parameter values to be set taking into account all relevant factors and variables.

FIG. 3B shows a corresponding system with a separate expert module 10, whereby expert module 10 is coupled with a ventilator 12 and an ECLS system 28. In this version, the expert module 10 is optionally equipped with a monitor 34, whereby settings of the ventilator 12 and the ECLS system 28 can be made or adjusted using the monitor 34, as shown schematically with the dashed lines. FIG. 3C also shows a version with an expert module 10 integrated in an ECLS system 28, whereby the ECLS system 28 also includes a monitor 34, for example integrated in a console. The actual values 36B of the ECLS system 28 are received directly via a common interface by implementing expert module 10 in the ECLS system 28.

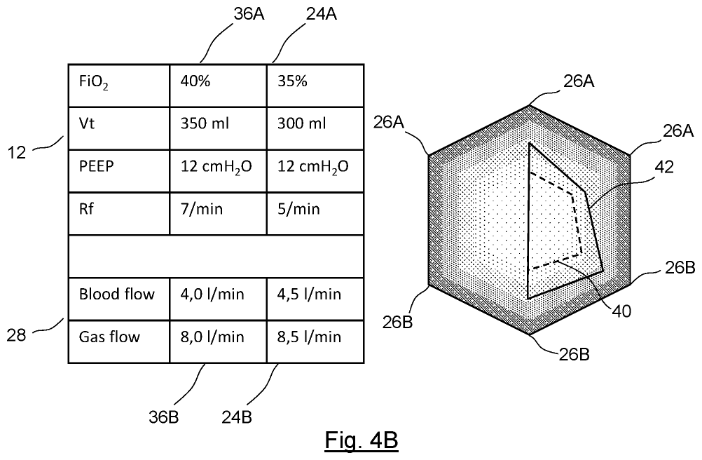

A specific example of measured vital parameters 16 and a course 22 as well as the respective support of a ventilator and an ECLS system is shown in FIG. 4A. In this example, four vital parameters 16 are received by the expert module, namely the expiratory oxygen fraction (FetO₂), the oxygen supply (DO₂), the oxygen uptake capacity (VO₂) and the carbon dioxide release (VCO₂). However, an alternative vital parameter 16 or an alternative number of vital parameters 16 can also be received.

The course 22 of the vital parameters 16 is shown as an example for the last 20 minutes in five-minute sections above the horizontal time axis, for example if previously measured values are no longer relevant for the current development of the clinical picture. The measured values of the vital signs 16 are displayed, in percentage or as ml/min, next to the total value (total, continuous line) separately for the specific part of the ventilator (Resp, semicolon line) on the one hand and the ECLS system (ECMO, dashed line) on the other hand, whereby the nominal ratio 38 or the part of the ventilator is also displayed as a value in percentage of the total value.

FIG. 4B shows a specific example of suggested 24A setpoints or setpoint values for ventilator 12 and ECLS system 28. The 24A setpoints or setpoint values for the ventilator in this example are the inspiratory oxygen fraction (FiO₂), respiratory volume (Vt), positive end expiratory pressure (PEEP) and respiratory frequency (Rf), wherein the 24B setpoints or setpoint values for the ECLS system include blood pump flow rate and gas volume flow rate. In addition to the suggested setpoints or setpoint values of 24A, 24B, the current actual values of 36A and 36B are also displayed, so that the physician can easily oversee the current state and any suggested adjustment of the values.

Furthermore, the effects or impact of the calculated setpoints or setpoint values 24A, 24B are also graphically shown on the right, whereby the relevant physiological factors 26A, 26B are shown in a polygon, here a hexagon. The physiological factors 26A, 26B are arranged in such a way that mutually determining factors are compared, with one point in the marginal area corresponding to a negative effect and one point in the central area corresponding to a positive effect on the physiological condition of the patient. The physiological factors 26A, 26B are presented in such a way that the upper physiological factors 26A are indicative of over-ventilation and the lower physiological factors 26B are indicative of ventilatory insufficiency. Such a design is only optional, but offers further simplification and increased clarity for the treating physician, as the effects or impact on the current therapy and the technical and physiological factors can be simultaneously presented and interpreted in an understandable way.

In the graphical representation, the effects are still shown as polygons, whereby both the effects of the current actual values 36A, 36B of the operating parameter values and the effects of the proposed setpoints or setpoint values 24A, 24B are shown separately and in different colors, as indicated by the reference signs 42 and 40 respectively. Thus, it is immediately apparent how an adjustment of the respective operating parameter values affects the physiological condition of the patient, so that the physician can adjust the treatment in real time with regard to the most relevant (because particularly critical for the patient) physiological factors 26A, 26B if necessary. A color identifier or a color gradient may also be used as a background in the graphical representation, as shown above with the different hatching, to indicate an improvement and/or a tolerance range for the respective physiological factor 26A, 26B and to further facilitate the interpretation of the effects of the setpoints or setpoint values 24A, 24B.

Where applicable, all the individual features depicted in the embodiments may be combined and/or exchanged without departing from the scope of the invention.

### REFERENCE CHARACTER LIST

10 Expert module

12 Ventilator

14 Patient

16 Vital signs

18 Evaluation unit

20 Target value

22 Course

24A,B Set point or setpoint value

26A,B Physiological factor

28 ECLS system or ECMO system

30 Signal

32 Physiological Model

34 Monitor

36A,B Actual value

38 Ratio

40 Effect of setpoints or setpoint values

42 Effect of actual values

1-42. (canceled)

43. An expert module for a ventilator of a patient, wherein the expert module is adapted to receive at least one vital parameter of the patient, and wherein the expert module comprises an evaluation unit adapted to:
store the at least one vital parameter for a predetermined period of time,
determine a target value of the at least one vital parameter based on a course of the at least one vital parameter and/or predetermined clinical data,
determine a setpoint or setpoint value of at least one operating parameter of the ventilator based on the course of the at least one vital parameter and the target value of the at least one vital parameter and depending on at least two physiological factors of the patient,
determine a setpoint or setpoint value of at least one operating parameter of an ECLS system coupled to the patient based on the course of the at least one vital parameter and the target value of the at least one vital parameter and depending on the at least two physiological factors, and
output a signal indicative of the setpoints or setpoint values of the at least one operating parameter of the ventilator and the at least one operating parameter of the ECLS system.

44. The expert module of claim 43, wherein the expert module is adapted to continuously receive the at least one vital parameter of the patient.

45. The expert module of claim 43, further adapted to receive actual values of the operating parameters from the ventilator and the ECLS system, the evaluation unit being adapted to determine the setpoints or setpoint values in dependence upon the actual values.

46. The expert module according to claim 43, wherein the ventilator is configured to assist spontaneous breathing of the patient or to provide artificial respiration of the patient.

47. The expert module according to claim 46, wherein the ventilator is a mechanical ventilator.

48. The expert module according to claim 43, wherein the expert module is coupled to a monitor, and wherein outputting the signal comprises a graphical representation of the setpoints or setpoint values of the at least one operating parameter of the ventilator and the at least one operating parameter of the ECLS system, the actual value, the target value, and/or the course of the corresponding value.

49. The expert module according to claim 48, wherein the evaluation unit determines the setpoints or setpoint values from a physiological model stored in the evaluation unit and wherein the signal further comprises a graphical representation of an impact of the setpoints or setpoint values of the at least one operating parameter of the ventilator and the at least one operating parameter of the ECLS system on the physiological factors modeled by the evaluation unit.

50. The expert module according to claim 49, wherein the impact is separate for the ventilator and the ECLS system and/or wherein the signal comprises, in addition to the impact of the setpoints or setpoint values of the at least one operating parameter of the ventilator and the at least one operating parameter of the ECLS system, a graphical representation of a modeled impact of the actual values on the physiological factors.

51. The expert module according to claim 43, wherein the physiological factors are not operating parameters of the ventilator.

52. The expert module according to claim 43, wherein the physiological factors represent the functionality of the patient's pulmonary or lung function.

53. The expert module according to claim 43, wherein the physiological factors are indicative of over-ventilation or ventilation insufficiency.

54. The expert module according to claim 53, wherein the physiological factors comprise one or more of mechanical pulmonary trauma, atrophy, barotrauma, volutrauma, alkalosis, oxygen toxicity, absorption atelectasis, acidosis, hypoxia, stress, and hemodynamic side effects.

55. The expert module according to claim 54, wherein the evaluation unit is adapted to determine the setpoints or setpoint values of the at least one operating parameter of the ventilator and the at least one operating parameter of the ECLS system in dependence on three or more physiological factors.

56. The expert module according to claim 55, wherein the evaluation unit is adapted to determine the setpoints or setpoint values of the at least one operating parameter of the ventilator and the at least one operating parameter of the ECLS system in dependence on five or six physiological factors.

57. The expert module according to claim 43, wherein the evaluation unit is adapted to determine the set points or setpoint values of the at least one operating parameter of the ventilator and the at least one operating parameter of the ECLS system from a physiological model stored in the evaluation unit such that a negative impact on the physiological factors is minimized and/or the physiological factors do not exceed a predetermined tolerance range.

58. The expert module according to claim 43, wherein the at least one vital parameter comprises one or more of pulsoximetric oxygen saturation, expiratory oxygen fraction, expiratory carbon dioxide fraction, oxygen uptake capacity, carbon dioxide release, and blood pH.

59. The expert module according to claim 43, wherein the expert module is adapted to receive at least two vital parameters.

60. The expert module according to claim 43, wherein the expert module is adapted to receive at least three or four vital parameters.

61. The expert module according to claim 43, wherein the at least one operating parameter of the ventilator includes the respiratory volume, peak inspiratory pressure, positive end expiratory pressure, respiratory frequency, inspiratory oxygen fraction, ratio between inhalation duration and exhalation duration, and/or inspiratory carbon dioxide fraction, and/or wherein the at least one operating parameter of the ECLS system includes blood pump flow rate, system pressure and/or gas volume flow.

62. The expert module according to claim 43, wherein the evaluation unit is adapted to determine the setpoint or setpoint value each of two, three, or four operating parameters of the ventilator and the setpoint or setpoint value for each of two operating parameters of the ECLS system.

63. The expert module according to claim 43, wherein the evaluation unit is further adapted to determine the setpoints or setpoint values for the at least one operating parameter of the ventilator and the at least one operating parameter of the ECLS system in dependence on a ratio of support of the ventilator to the ECLS system determined by the evaluation unit and related to the at least one vital parameter.

64. The expert module according to claim 43, wherein the signal further comprises a request to receive at least one further vital parameter.

65. The expert module according to claim 43, wherein the evaluation unit is adapted to compare the course of the at least one vital parameter with a tolerance range and/or a modeled course and to output the signal as an alarm, once a deviation of the at least one vital parameter exceeding a predetermined threshold or limit value is detected.

66. A ventilator comprising:
an expert module adapted to receive at least one vital parameter of the patient, and wherein the expert module comprises an evaluation unit adapted to:
store the at least one vital parameter for a predetermined period of time,
determine a target value of the at least one vital parameter based on a course of the at least one vital parameter and/or predetermined clinical data, and
determine a setpoint or setpoint value of at least one operating parameter of the ventilator based on the course of the at least one vital parameter and the target value of the at least one vital parameter and depending on at least two physiological factors of the patient,
determine a setpoint or setpoint value of at least one operating parameter of an ECLS system coupled to the patient based on the course of the at least one vital parameter and the target value of the at least one vital parameter and depending on the at least two physiological factors, and
output a signal indicative of the setpoints or setpoint values of the at least one operating parameter of the ventilator and the at least one operating parameter of the ECLS system.

67. The ventilator of claim 66, wherein the ventilator is a mechanical ventilator.

68. The ventilator according to claim 66, comprising a device for detecting the at least one vital parameter of the patient, the device for detecting the at least one vital parameter of the patient being communicatively coupled to the expert module.

69. An ECLS system comprising:
an expert module adapted to receive at least one vital parameter of the patient, wherein the expert module comprises an evaluation unit adapted to:
store the at least one vital parameter for a predetermined period of time,
determine a target value of the at least one vital parameter based on a course of the at least one vital parameter and/or predetermined clinical data, and
determine a setpoint or setpoint value of at least one operating parameter of a ventilator based on the course of the at least one vital parameter and the target value of the at least one vital parameter and depending on at least two physiological factors of the patient,
determine a setpoint or setpoint value of at least one operating parameter of the ECLS system coupled to the patient based on the course of the at least one vital
parameter and the target value of the at least one vital parameter and depending on the at least two physiological factors, and
output a signal indicative of the setpoints or setpoint values of the at least one operating parameter of the ventilator and the at least one operating parameter of the ECLS system.

70. The ECLS system of claim 69, wherein the ECLS system is an ECMO system.

71. The ECLS system according to claim 69, further comprising a device for detecting the at least one vital parameter of the patient, the device for detecting the at least one vital parameter of the patient being communicatively coupled to the expert module.

72. A system comprising:
a ventilator comprising:
an expert module adapted to receive at least one vital parameter of the patient, wherein the expert module comprises an evaluation unit adapted to:
store the at least one vital parameter for a predetermined period of time,
determine a target value of the at least one vital parameter based on a course of the at least one vital parameter and/or predetermined clinical data, and
determine a setpoint or setpoint value of at least one operating parameter of the ventilator based on the course of the at least one vital parameter and the target value of the at least one vital parameter and depending on at least two physiological factors of the patient,
determine a setpoint or setpoint value of at least one operating parameter of an ECLS system coupled to the patient based on the course of the at least one vital parameter and the target value of the at least one vital parameter and depending on the at least two physiological factors, and
output a signal indicative of the setpoints or setpoint values of the at least one operating parameter of the ventilator and the at least one operating parameter of the ECLS system; and
an ECLS system.

73. A method comprising:
receiving at least one vital parameter of a patient from an expert module;
storing the at least one vital parameter for a predetermined period of time in the expert module;
determining a target value of the at least one vital parameter based on a course of the at least one vital parameter and/or predetermined clinical data in the expert module;
determining a setpoint or setpoint value of at least one operating parameter of a ventilator to be provided for the patient in the expert module, wherein the setpoint or setpoint value is determined based on the course of the at least one vital parameter and the target value of the at least one vital parameter and in dependence on at least two physiological factors of the patient;
determining a setpoint or setpoint value of at least one operating parameter of an ECLS system to be provided for the patient in the expert module, wherein the setpoint or setpoint value is determined based on the course of the at least one vital parameter and the target value of the at least one vital parameter and in dependence on the physiological factors; and
outputting by the expert module a signal indicative of the setpoints or setpoint values of the operating parameters of the at least one operating parameter of the ventilator and the at least one operating parameter of the ECLS system.

74. The method according to claim 73, wherein the signal indicates a proposed use of the ventilator and/or the ECLS system.

75. The method according to claim 73, wherein determining the target value and/or the setpoints or setpoint values of the at least one operating parameter of the ventilator and the at least one operating parameter of the ECLS system is continuous or periodic.

76. The method according to claim 73, wherein the expert module re-determines the target value and/or the setpoints or setpoint values of the at least one operating parameter of the ventilator and the at least one operating parameter of the ECLS system after a manual adjustment of the operating parameter values.

77. The method according to claim 73, wherein the expert module further receives actual values of the operating parameters from the ventilator and/or the ECLS system and determines the setpoints or setpoint values of the at least one operating parameter of the ventilator and the at least one operating parameter of the ECLS system in dependence on the actual values.

78. The method according to claim 73, wherein the signal is output as a graphical representation of the setpoints or setpoint values of the at least one operating parameter of the ventilator and the at least one operating parameter of the ECLS system, the actual value, the target value, and/or the course of the corresponding value on a monitor communicatively coupled to the expert module.

79. The method according to claim 78, wherein the setpoints or setpoint values of the at least one operating parameter of the ventilator and the at least one operating parameter of the ECLS system are determined from a physiological model stored in the expert module, and wherein the signal further comprises a graphical representation of an impact of the setpoints or setpoint values of the at least one operating parameter of the ventilator and the at least one operating parameter of the ECLS system on the physiological factors modeled by the expert module.

80. The method according to claim 79, wherein the representation of the impact is carried out separately for the ventilator and the ECLS system and/or wherein the signal comprises, in addition to the impact of the setpoints or setpoint values of the at least one operating parameter of the ventilator and the at least one operating parameter of the ECLS system, a graphical representation of a modeled impact of the actual values on the physiological factors.

81. The method according to claim 73, wherein the physiological factors are not operating parameters of the ventilator.

82. The method according to claim 73, wherein the physiological factors represent a functionality of the patient's pulmonary or lung function.

83. The method according to claim 73, wherein the physiological factors are indicative of over-ventilation or ventilation insufficiency.

84. The method according to claim 73, wherein, upon determining a positive impact on at least one physiological factor, the impact on at least one other physiological factor is calculated, simulated, or modelled.

85. The method according to claim 73, wherein the setpoints or setpoint values of the at least one operating parameter of the ventilator and the at least one operating parameter of the ECLS system are determined on the basis of a physiological model stored in the expert module in such a way that a negative impact on the physiological factors is minimized and/or the physiological factors do not exceed a predetermined tolerance range.

86. The method according to claim 73, wherein the setpoints or setpoint values of the at least one operating parameter of the ventilator and the at least one operating parameter of the ECLS system are determined in dependence on a ratio, determined by the expert module, of a support intensity by the ventilator in relation to a support intensity by the ECLS system in relation to the at least one vital parameter.

87. The method according to claim 73, wherein the signal further comprises a request to receive at least one further vital parameter.

88. The method according to claim 73, wherein the course of the at least one vital parameter is compared with a tolerance range and/or a modeled course in the expert module and wherein an alarm is output, once a deviation of the at least one vital parameter exceeding a predetermined threshold value or limit value is detected.

89. The method according to claim 73, wherein the expert module delivers an alarm, once a manual setting of the operating parameter values exceeds a predetermined threshold value or limit value of the operating parameter values, the at least one vital parameter, and/or the modeled impact on the physiological factors.
